(19) United States
(12) Patent Application Publication
(10) Pub. No.: US 2013/0323334 A1
(43) Pub. Date: Dec. 5, 2013

(54) Use Of Compositions For Preventing Chemotherapy And Radiotherapy Induced Alopecia (CRIA), Reducing CRIA Impact And Improving The Appearance Of Hair Re-Growth After CRIA

(71) Applicant: Saad Harti, Lutry (CH)

(72) Inventor: Saad Harti, Lutry (CH)

(73) Assignee: LEGACY HEALTHCARE LTD., Valleta (MT)

(21) Appl. No.: 13/985,958

(22) PCT Filed: Feb. 22, 2012
(86) PCT No.: PCT/EP2012/052992

§ 371 Date: Aug. 16, 2013

Related U.S. Application Data
(60) Provisional application No. 61/445,601, filed on Feb. 23, 2011.

(51) Int. Cl. A61K 36/185; A61K 36/88; A61K 36/77; A61K 36/752

(52) U.S. Cl. CPC A61K 36/185; A61K 36/752; A61K 36/88; A61K 36/77
USPC 424/736

(57) ABSTRACT

Compositions administered by topical route contain as active ingredients an extract of Allium species, an extract of Citrus species, an extract of Paullinia species and an extract of Theobroma species, for preventing CRIA, reducing CRIA impact and improving the appearance of hair re-growth after CRIA. A method for preventing, reducing impact and improving the appearance of hair re-growth on a human head before, during and after CRIA, said method comprising the administration by topical route of a composition containing as active ingredient an extract of Allium species, an extract of Citrus species, an extract of Paullinia species and an extract of Theobroma species.

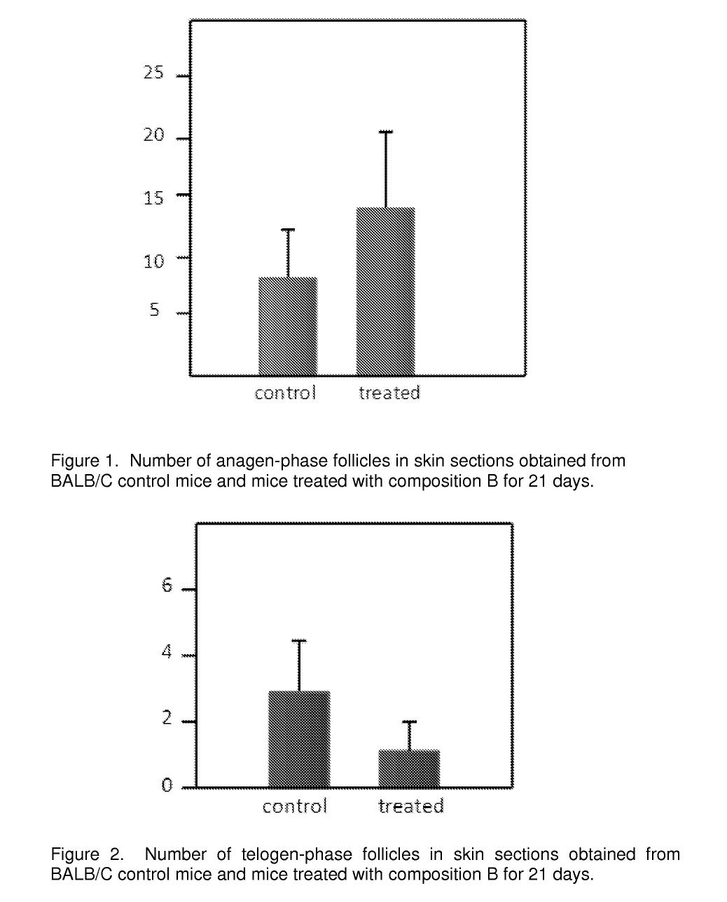

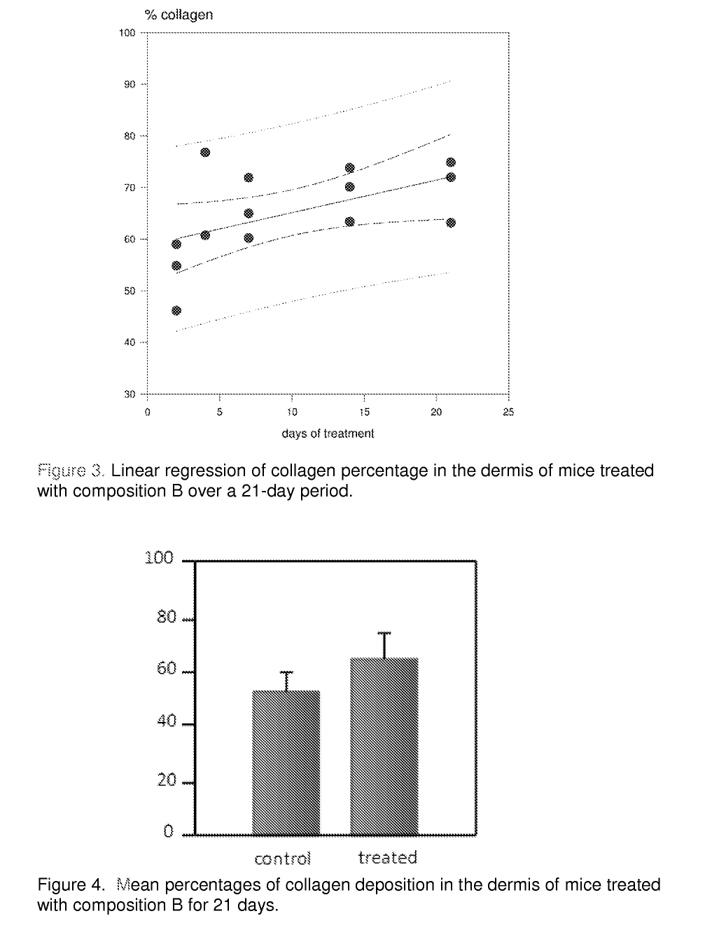

### BACKGROUND AND SUMMARY

The invention concerns a new use of compositions containing as active ingredient an extract of Allium species, an extract of Citrus species, an extract of Paullinia species and an extract of Theobroma species, preventing CRIA (Chemotherapy and Radiotherapy Induced Alopecia), reducing CRIA impact and improving the appearance of hair re-growth after CRIA , and methods thereof.

From patent application WO 2008/113912 it is known that, compositions containing as active ingredient an extract of Allium species, an extract of Citrus species and
either an extract of Paullinia species and an extract of Theobroma species
or an extract of Salix species and zinc sulphate increase the hair growth.

At the cellular level, CRIA (Chemotherapy and Radiotherapy Induced Alopecia) originates from the premature on-set of the catagen phase of the hair due to the destruction (apoptosis) by a chemotherapy agent of the cells in the hair follicles, especially the cells linking the hair to the dermal papilla.

The catagen phase consists in the involution of the hair follicle and beginning of detachment of the hair from the dermal papilla, due to the entry into massive apoptosis (programmed cell death) of the cells in the hair follicles, in particular the cells linking the hair to the dermal papilla.

At the molecular level, previous studies using a mouse chemotherapy induced alopecia model have shown that p53, a pro-apoptotic and tumor suppressor protein, played essential roles in mediating anticancer agent-induced apoptosis in hair follicles.

Such an apoptosis-promoting effect may involve numerous p53 responsive genes acting through different mechanisms. For instance, p53 up-regulates transcription of Bax and down-regulates transcription of Bcl-2, thus decreasing the Bcl-2/Bax ratio and predisposing cells to apoptosis. It has also been suggested that p53 is involved in mediating DNA damage responses in the hair follicles induced by chemotherapeutic agents, hence in the control of hair regression.

Interestingly, pharmacological or genetic suppression of p53 in mice greatly reduced or prevented chemotherapy-induced apoptosis in hair matrix keratinocytes and hair loss.

The above-mentioned investigational work indicates that inhibition of pro-apoptotic protein or re-establishment of the balance of intracellular levels of pro- and anti-apoptotic proteins (such as p53 and Bcl-2) in hair follicles may serve as an effective treatment for preventing CRIA, reducing CRIA impact and improving the appearance of hair re-growth after CRIA.

It has been shown that application of compositions containing as active ingredient an extract of Allium species, an extract of Citrus species, an extract of Paullinia species and an extract of Theobroma species, enables the increase of the intracellular BCL2 (an anti-apoptotic protein) to its normal level, hence enabling the cells in the hair follicles, specially the cells linking the hair to the dermal papilla not to enter into premature apoptosis, as provoked by the chemotherapy agent.

The delay of the premature on-set of the catagen phase enables the hair remain in its anagen phase longer, and therefore not fall because of the action of the chemotherapy agent.

Advances in cancer treatment have enabled patients to live longer, more productive lives and to continue working and enjoying normal social and recreational activities. The most visible and emotionally distressing side effect of cancer therapy is Chemotherapy and Radiotherapy Induced Alopecia (CRIA). Partial or total hair loss is a constant source of stress for the patient and a public display of underlying disease. Clinical studies of women undergoing cancer treatment consistently identify alopecia as the most traumatic side effect of chemotherapy, associated with low self-esteem, poor body image, and diminished quality of life. Men are also profoundly affected by CRIA , but there are few support groups or educational/assistive services available to help male chemotherapy and radiotherapy patients cope with the psychologically damaging effects of hair loss.

CRIA occurs when rapidly growing and dividing cell populations in anagen phase follicles are damaged by the systemic chemotoxic agents or radiotherapy killing actively growing tumor cells. CRIA is a temporary condition; hair growth for the majority of patients resumes when damaged follicles remodel and re-enter anagen phase. However, restoration of the normal anagen to telogen ratio of 9:1 can take from six months to one year. From the perspective of hair cycle biology, an effective treatment for CRIA would protect susceptible, rapidly growing bulb matrix cells from the destructive effects of systemic chemotherapy agent(s). Alternatively, CRIA patients could benefit from a product that would rapidly induce normal hair growth by accelerating the synchronized transition of damaged follicles from telogen to anagen phase to restore the normal anagen to telogen ratio and cosmetically normal hair.

It has been discovered that the administration by topical route of a composition containing as active ingredient an extract of Allium species, an extract of Citrus species, an extract of Paullini species and an extract of Theobroma species has a novel and unexpected effect preventing CRIA, reducing CRIA impact and improving the appearance of hair re-growth after CRIA. Thus the present invention concerns a new use of compositions administered by topical route, containing as active ingredient an extract of Allium species, an extract of Citrus species, an extract of Paullini species and an extract of Theobroma species, for preventing CRIA, reducing CRIA impact and improving the appearance of hair re-growth after CRIA.

### DETAILED DESCRIPTION

A first observation has been made during a study with depilated mice. The study was conducted as a model of chemotherapy induced anagen effluvium to evaluate the efficacy of a topical active ingredient containing an extract of Allium species, an extract of Citrus species, an extract of Paullini species and an extract of Theobroma species, to accelerate the rate of hair re-growth. In addition, one case report of a female patient undergoing chemotherapy seems to support animal observations.

Another object of the invention is also a new use of topical compositions containing an aqueous-alcoholic extract of Allium species, an aqueous-alcoholic extract of Citrus species, an aqueous alcoholic extract (atomised or not) of Paullini species and an aqueous-alcoholic extract (atomised or not) of Theobroma species, for preventing CRIA, reducing CRIA impact and improving the appearance of hair re-growth after CRIA. Most preferably, the compositions are used before, during and after CRIA.

Among the new use for preventing CRIA, reducing CRIA impact and improving the appearance of hair re-growth after CRIA, according to the invention, those which are of more particular interest are those in which the preferred topical composition contains from 65% to 93% of an aqueous-alcoholic extract of Allium species, from 5% to 33% of an aqueous-alcoholic extract of Citrus species, from 0.25% to 2.5% of an aqueous alcoholic extract (atomised or not) of Paullinia species and from 0.25% to 2.5% of an aqueous-alcoholic extract (atomised or not) of Theobroma species. Among the methods for preventing CRIA, reducing CRIA impact and improving the appearance of hair re-growth after CRIA, according to the invention, those which are the more preferred interest are the methods in which the compositions are containing from 65% to 93% of an aqueous-alcoholic extract of Allium species, from 5% to 33% of an aqueous-alcoholic extract of Citrus species, from 0.25% to 2.5% of an aqueous-alcoholic extract (atomised or not) of Paullinia species and from 0.25% to 2.5% of an aqueous-alcoholic extract (atomised or not) of Theobroma species and especially those containing from 65% to 93% of an aqueous-alcoholic extract of Allium cepa, from 5% to 33% of an aqueous-alcoholic extract of Citrus lemon, from 0.25% to 2.5% of an aqueous-alcoholic extract (atomised or not) of Paullinia species and from 0.25% to 2.5% of an aqueous-alcoholic extract (atomised or not) of Theobroma species.

The term extract of Allium species or aqueous-alcoholic extract of Allium species refers particularly to aqueous-alcoholic extracts and native extracts obtained from all species of the genus Allium (family Liliaceae) and especially Allium cepa. The term extract of Citrus species or aqueous-alcoholic extract of Citrus species refers particularly to aqueous-alcoholic extracts and native extracts obtained from all species of the genus Citrus (family Rutaceae) and especially Citrus lemon. The term extract (atomised or not) of Paullinia species or aqueous-alcoholic extract (atomised or not) of Paullinia species refers particularly to aqueous-alcoholic extracts and native extracts obtained from all species of the genus Paullinia (family Sapindaceae) and especially Paullinia cupana. The term extract (atomised or not) of Theobroma species or aqueous-alcoholic extract (atomised or not) of Theobroma species refers particularly to aqueous-alcoholic extracts and native extracts obtained from all species of the genus Theobroma (family Malvaceae) and especially Theobroma cacao.

The most preferred compositions used according to the invention are those containing approximately 87% of an aqueous-alcoholic extract of Allium cepa, approximately 12% of an aqueous-alcoholic extract of Citrus lemon, approximately 0.5% of an aqueous-alcoholic extract (atomised or not) of Paullinia cupana and approximately 0.5% of an aqueous-alcoholic extract (atomised or not) of Theobroma cacao;

These compositions are prepared as indicated in patent application WO 2008/113912, which is incorporated by reference herein. These cosmetical or pharmaceutical compositions are prepared by conventional methods, in which inert, organic or inorganic excipients are added to the compositions obtained according to the invention.

According to the invention the composition is administered daily, before the chemotherapy or radiotherapy, during the chemotherapy or radiotherapy , and up to six months after chemotherapy or radiotherapy has ended, with a composition containing as active ingredient an extract of Allium species, an extract of Citrus species, an extract of Paullinia species and an extract of Theobroma species.

In order to obtain a cosmetically satisfying effect on the hair growth, it is necessary to perform the administration of the compositions during up to 6 months after the chemotherapy or radiotherapy treatment has ended. When using the compositions obtained according to the invention, doses may vary within relatively wide limits and must be set according to the person being treated and the condition concerned. Pharmaceutical compositions normally contain from 0.2 to 500 mg, preferably from 1 to 200 mg, of active ingredients as defined above, in the form of dry extract.

Based on previous observations, we hypothesized that the compositions according to the invention might effectively suit CRIA patients, whose follicles are prematurely forced into telogen phase by many chemotherapy agents, causing widespread hair loss. A mouse depilation model was used to simulate chemotherapy-induced anagen effluvium to determine whether this topical biotherapy could accelerate the rate of hair growth in a chemotherapy induced alopecia model and/or protect hair follicles in chemotherapy-affected scalp from undergoing abnormal apoptosis-driven regression (catagen followed by telogen phase). This will provide support for clinical evaluation of the topical extract as an easy-to-use, natural solution for preventing CRIA, reducing CRIA impact and improving the appearance of hair re-growth after CRIA.

### Examples of Compositions

As a drug

### Hair Lotion

| | |
|---|---|
| Active ingredient: | Composition A 40 g per 100 ml |
| Excipients: | aqueous ethanol at 55° |
| Treatment: | Application 1-2 times daily friction |

### A Cosmetics

### 1°) Hair Lotion

| | |
|---|---|
| Active ingredient: | Composition A 21 g per 100 ml |
| Ingredients: | aqua, alcohol, betaine, glycerin, |
| | polysorbate 20, maltodextrin, silica. |
| Treatment: | Application 1 to 2 times daily friction. |

2°) Hair Gel

| | |
|---|---|
| Active ingredient: | Composition A 21% |
| Excipients: | 96% ethanol (10%), betaine 2%, 5% glycerol, |
| | Ultragel 300 (1%), Sterile demineralized |
| | water qs 100%. |
| Treatment: | Application 1 to 2 times daily massage. |

3°) Shampoo

| | |
|---|---|
| Active ingredient: | Composition A 10% |
| Excipients: | sodium dodecyl sulfate at 25% (10%), sodium |
| | chloride, 3% citric acid 0.1%, 0.5% |
| | imidazolydinylurée, demineralized water |
| | sterile qs 100% |
| Treatment: | 1 shampoo every day. |

4°) After Shampoo

| | |
|---|---|
| Active ingredient: | Composition A 20% |
| Excipients: | Montanov 68 (5%), DM Amonyl 2%, 3% |
| | dimethicone, imidazolydinylurée 0.5%, |
| | sterile demineralized water qs 100%. |
| Treatment: | 1 shampoo every day. |

1. A method for preventing CRIA, reducing CRIA impact and improving the appearance of hair re-growth after CRIA, the method comprising administering by topical route to a subject in need thereof of a composition containing as active ingredients an extract of Allium species, an extract of Citrus species, an extract of Paullinia species and an extract of Theobroma species.

2. The method for preventing, reducing impact and improving the appearance of hair re-growth according to claim 1, for patients before, during and after CRIA, wherein the composition further comprises an aqueous-alcoholic extract of Allium species, an aqueous-alcoholic extract of Citrus species, an aqueous alcoholic extract (atomised or not) of Paullinia species and an aqueous-alcoholic extract (atomised or not) of Theobroma species.

3. The method according to claim 1, wherein the composition further comprises from 65% to 93% of an aqueous-alcoholic extract of Allium species, from 5% to 33% of an aqueous-alcoholic extract of Citrus species, from 0.25% to 2.5% of an aqueous alcoholic extract of Paullinia species and from 0.25% to 2.5% of an aqueous-alcoholic extract of Theobroma species.

4. The method according to claim 1, further comprising administering the composition daily during a period of several months.

5. The method according to claim 1, wherein the composition further comprises from 0.2 to 500 mg in the form of a dry extract.

6. The method according to claim 5, wherein the composition further comprises from 1 to 200 mg in the form of a dry extract.

7. The method according to claim 1, further comprising preventing, reducing impact and improving the appearance of hair regrowth on a human head/scalp before, during and after CRIA.
